(19) United States
(12) Patent Application Publication
(10) Pub. No.: US 2017/0138828 A1
(43) Pub. Date: May 18, 2017

(54) METHOD OF SOIL LIQUEFACTION TESTING AND REMEDIATION

(71) Applicant: Jonathan Scott Ellington, Charleston, SC (US)

(72) Inventor: Jonathan Scott Ellington, Charleston, SC (US)

(21) Appl. No.: 15/353,384

(22) Filed: Nov. 16, 2016

Related U.S. Application Data
(60) Provisional application No. 62/256,294, filed on Nov. 17, 2015.

(51) Int. Cl. G01N 3/24; G01N 33/24

(52) U.S. Cl. CPC G01N 3/24; G01N 33/24; E02D 7/00

(57) ABSTRACT

A method of soil liquefaction testing and remediation and devices for accomplishing the method are described. After determining that a soil deposit on a site is potentially subject to liquefaction, the soil deposit is subjected to mechanically produced shear strain reversals. Excess pore pressure generations are monitored at multiple depths and at multiple locations at the site, during application of the mechanically produced shear strain reversals, to determine liquefaction susceptibility of the soil deposit. Multiple ground improvements are positioned on the site mitigate liquefaction in those soils that are determined to be susceptible to liquefaction.

Applicant claims the benefit of U.S. Provisional Patent Application filed Nov. 17, 20, 2015, Ser. No. 62/256,297.

### FIELD OF THE INVENTION

This invention relates generally to the liquefaction testing and remediation of potentially liquefiable soils to minimize damage to supported structures and works from earthquake-induced liquefaction.

### BACKGROUND OF THE INVENTION

During and after an earthquake (seismic event), energy, in the form of compression, shear and Rayleigh waves, travels radially from the epicenter (focus or origin) of the earthquake in all directions. Large earthquakes are capable of generating elastic stress waves that carry destructive amounts of energy for hundreds of miles.

When stress waves travel through the earth's crust (bedrock), they result in repeated shearing deformations in overlying soils. The resulting shearing deformations are the principal cause of liquefaction in saturated cohesionless (sandy) soils.

When loose cohesionless (sandy) soils are subjected to repeated shear strain reversals (ground shaking back and forth), there is an accompanying decrease in volume of the soils. This is not unlike shaking loose marbles in a jar. The continued shaking of the jar will cause the loose marbles to “collapse” into a tighter nesting/packing.

If the subject sandy soils are saturated, that is, all pore/void space in the soil skeleton is substantially filled with groundwater, and unable to drain, each successive shear strain reversal causes a reduction in the volume of sand, and an accompanying increase in water pressure in the pore space. As the earthquake shaking continues, excess generated pore pressure increases as the soil attempts to shake into a tighter packing.

Geotechnically speaking, the total stress in a cohesionless soil is the sum of its effective stress (grain-to-grain contact pressure) and the pore water pressure in its void space. As pore water pressure increases due to the continued earthquake shaking, grain-to-grain contact pressure (effective stress) of the soil decreases. When the grain-to-grain contact pressure of the soil approaches zero, the mass of soil may lose all shear strength and behave as a semi-fluid. This phenomenon is known as liquefaction, and is responsible for extensive damage in earthquakes.

By definition, the phenomenon of cyclic mobility liquefaction occurs in cohesionless, saturated soils subjected to repeated shear strain reversals. This is generally the case with deposits of loose to medium density sandy soils accompanied by a shallow groundwater table in seismically active areas of the world.

Once a soil has liquefied and lost all shear strength, the deposit behaves as a semi-fluid. The liquefied deposit is often unable to support the loads imposed on it from structures and works. Historically, liquefaction has resulted in catastrophic bearing capacity failures.

Maximum credible earthquake events are generally defined by their Local or Moment Magnitudes (M_{L} or M_{W}), potential peak ground accelerations (PGAs) and the time duration of ground shaking. At present, the estimation of the liquefaction potential of soils is based empirically on the performance of soils with “similar” index properties during historical earthquakes in other locations around the globe.

Because of the relative unpredictability of earthquakes, universities, research institutes and government entities have to rely on stochastic methods to estimate the likelihood of the occurrence of seismic events of varying energies over varying time periods. Attempting to design for all earthquakes is infeasible. Building authorities and engineers must establish the maximum credible design seismic event that has a specific likelihood of occurring during the design-life of the building or work.

### SUMMARY OF THE INVENTION

A method of soil liquefaction testing and remediation and devices for accomplishing the method are described. Potentially liquefiable soil deposits are subjected to mechanically produced shear strain reversals. Excess pore water pressure generation is monitored at multiple depths and at multiple locations at the site during application of the mechanically produced shear strain reversals to determine actual liquefaction susceptibility of the soil deposit. Multiple ground improvements are positioned on the site mitigate liquefaction in those soils that are determined to be susceptible to liquefaction.

After installing ground improvements, the site may be subjected to additional mechanically produced shear strain reversals. Excess pore pressure generations are monitored during the application of the mechanically produced shear strain reversals to monitor the site's response to ground improvement. Additional site remediation may be performed if indicated by the additional shear strain reversals.

### BRIEF DRAWING DESCRIPTION

FIG. 1 demonstrates positioning of an energy producing mechanical excitation source positioned at depth in a soil deposit at a site.

FIG. 2 demonstrates an embodiment of an energy producing mechanical excitation source positioned at or near the surface of the soil deposit.

FIG. 3 is an exemplary depiction of soil layers, including liquefiable soil layers.

FIG. 4 shows ground improvement by drainage installation.

FIG. 5 shows ground improvement by soil densification.

FIG. 6a shows installation of a liquefaction sensor in a bore hole.

FIG. 6b shows installation of a liquefaction sensor in the soil by direct push.

### DESCRIPTION OF PREFERRED EMBODIMENTS

The apparatus and method of the present invention for testing and subsequent remediation of liquefiable soil for earthquake liquefaction protection for a structure or work thereon involves instrumenting a potentially liquefiable soil deposit with equipment capable of measuring pore water pressure generation/dissipation and shear strain reversals in-situ. Determining that a soil deposit at a site is potentially subject to liquefaction may be based on assumed or estimated soil index properties. The soil deposit is subjected to mechanically-produced shear strain reversals (soil accelerations) in excess of those anticipated from the design seismic event for durations longer than the design seismic event. The shear strain reversals may be produced at varying depths.

The present invention allows the subject site to be tested and treated according to soil accelerations of varying design earthquakes, (e.g. Moment Magnitudes, M_{W}, ranging from 5.0 to 9.5). The soil accelerations may be chosen for any magnitude of earthquake for a particular subject site. The subject site may be tested under varying shear strain reversals (soil accelerations). Pore pressure in the soil deposit is monitored during shear strain reversals. The testing equipment may be constructed and arranged to measure in-situ pore pressures and soil accelerations in one, two, or three dimensions. The actual liquefaction susceptibility of the soil deposit, as well as individual soil layers throughout the soil deposit, is determined by observation of the increase in pore pressure in the soil deposit as a result of subjecting the soil deposit to the soil accelerations (shear strain reversals).

The soil deposit is subjected to soil accelerations that produce shear strain reversals. The soil accelerations to which the soil deposit is subjected are determined according to the design earthquake chosen or required at the site. Published data is available to indicate the accelerations required for a particular design earthquake of a chosen magnitude.

If the soil deposit is found to be subject to liquefaction due to pore pressure increases resulting from shear strain reversals, subsequent treatment of the site is preferred to be performed. Since actual susceptibility to liquefaction is determined according to the method of the invention, the treatment applied to the soil deposit may be specifically determined, which frequently reduces the cost of treatment. Treatment is applied as a result of actual observations regarding liquefaction, rather than based on assumed and/or estimated index properties of the subject soil.

Treatment may include installing vertical drainage elements in a pattern determined from data gained by initial testing. Mechanical equipment may then be used as before to generate repeated shear strain reversals (soil accelerations) equivalent to, or greater than, those anticipated during the design earthquake through the full depth of the liquefiable soils, while again measuring pore pressure generation and dissipation during the application of shear strain reversals.

The spacing and frequency of the mechanically induced excitation points and any accompanying ground improvement (densification/positive drainage elements) may be determined and continually altered or revised across the soil deposit so that, upon completion of the improvement (1) all liquefiable soils present across the proposed treatment area have been subjected to accelerations equal to or greater than those of the design earthquake, and preferably, for a period longer than that anticipated from the design earthquake; and (2) all final measured excess generated pore pressures are held to levels less than those at which the onset of liquefaction and/or intolerable settlements occur.

In effect, the soils at the subject site are tested for actual liquefaction potential under shear strain reversals that are preferably in excess of those anticipated from an earthquake upon which the design earthquake is based. If indicated, the appropriate level of ground improvement to prevent any subsequent liquefaction during a design earthquake event is provided to the soil.

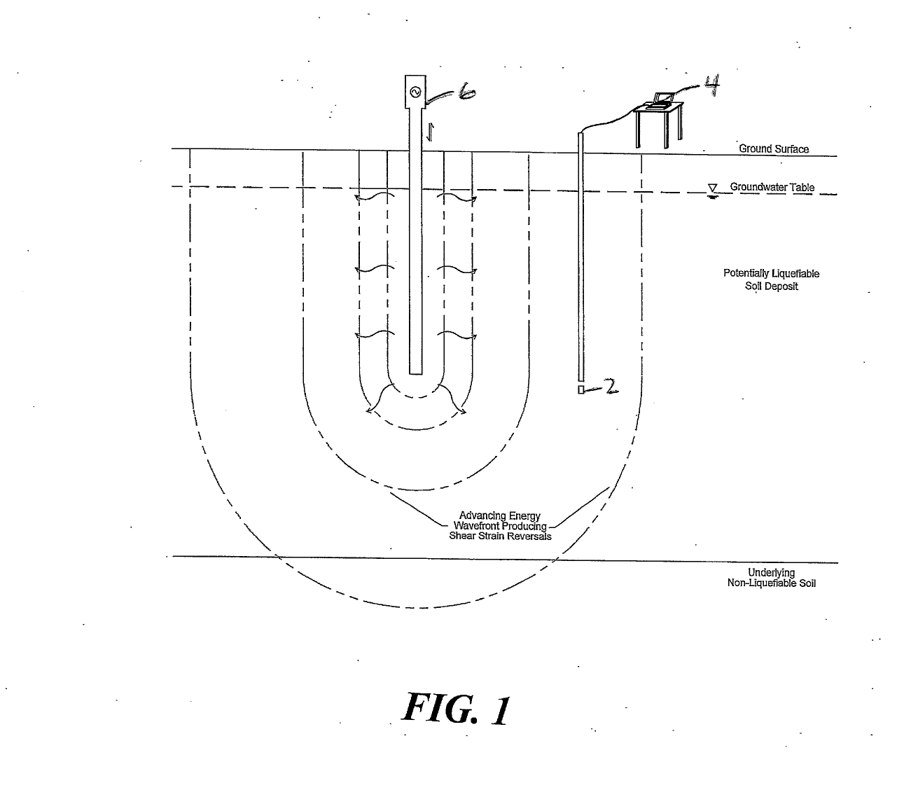

FIG. 1 is an overview of an embodiment of an initial site testing method for the evaluation of liquefaction throughout a potentially liquefiable deposit. FIG. 1 illustrates propagation of energy waves from a mechanical cyclic excitation source 6. The mechanical cyclic excitation source is preferably a vibratory device that vibrates the soil to produce the energy waves, such as a mechanical hammer. This source creates shear strain reversals that propagate radially from the source and through the surrounding potentially liquefiable soils.

A liquefaction sensor array transmits information related to measured shear strain reversals (displacements/velocities/accelerations) and pore water pressures during testing/evaluation to the data acquisition system. One or more liquefaction sensors, such as liquefaction sensor 2, are spaced apart from the mechanical cyclic excitation source 6. In this embodiment, the liquefaction sensor is positioned below the water table and at a depth in the soil and at a distance from the mechanical excitation source that are deemed to be appropriate for collecting information about the liquefaction potential of the subject soil. Data acquisition equipment 4 is used to collect information from the liquefaction sensor array. The information so acquired is used for evaluating whether soils at the site are liquefiable under the design earthquake. If the soils are determined to be liquefiable due to the induced increase in pore pressure from shear strain reversals, appropriate soil treatment is designed. The method of the invention indicates which soil layers should be considered for treatment. The testing equipment is constructed and arranged to measure in-situ pore pressures in a range of 0.0 to 100.0 pounds per square inch (psi).

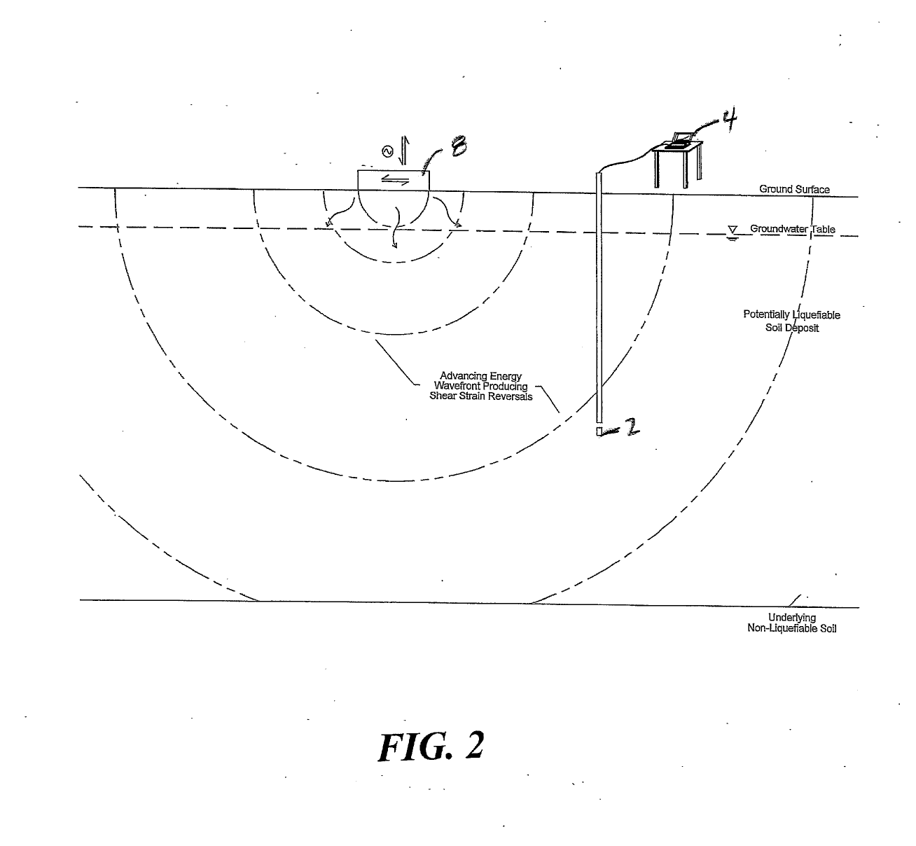

FIG. 2 is an overview of the process described in FIG. 1, but employing an alternate mechanical excitation source 8 for the creation and propagation of energy waves. The mechanical excitation source is positioned at, or only slightly below, the ground surface, and above the water table. The mechanical excitation source is preferred to be a vibratory device that produces the required vibrations according to the design earthquake. Shear strain reversals propagate radially from the source through the potentially liquefiable soils. Information is collected as described with regard to FIG. 1.

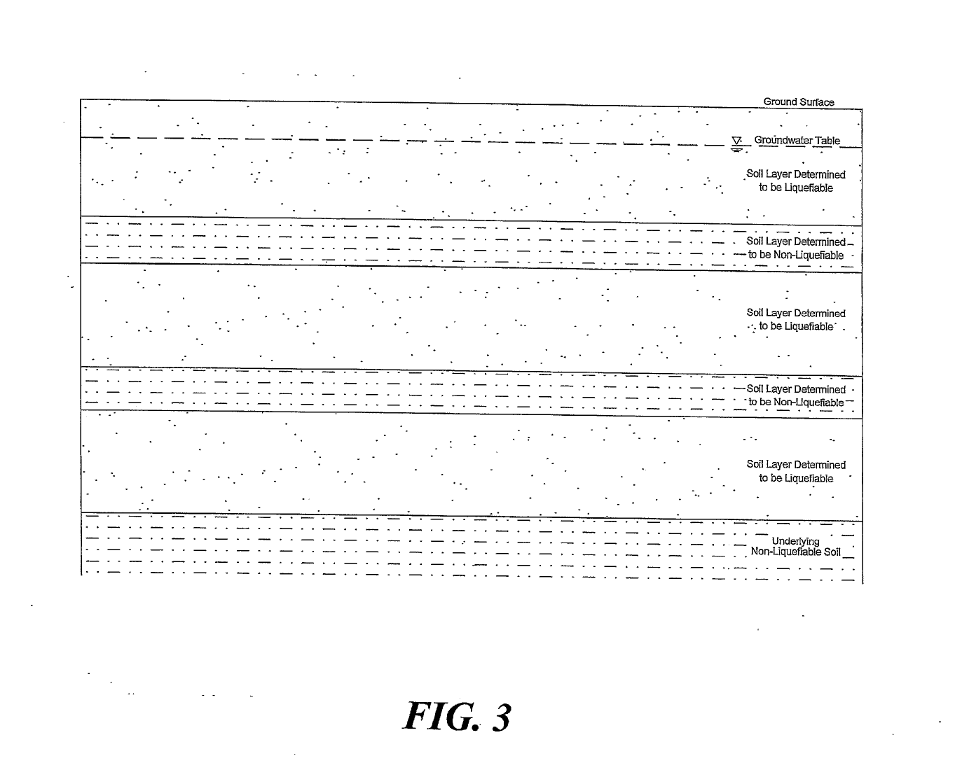

FIG. 3 is an example of the post-testing characterization of a site that was subjected to shear strain reversal as described herein. The post-testing characterization is based upon an evaluation of data collected from shear strain reversal and pore water pressure data collected in-situ from the liquefaction sensor array. Various layers are shown to be liquefiable or non-liquefiable. Treatment decisions may be made from this information.

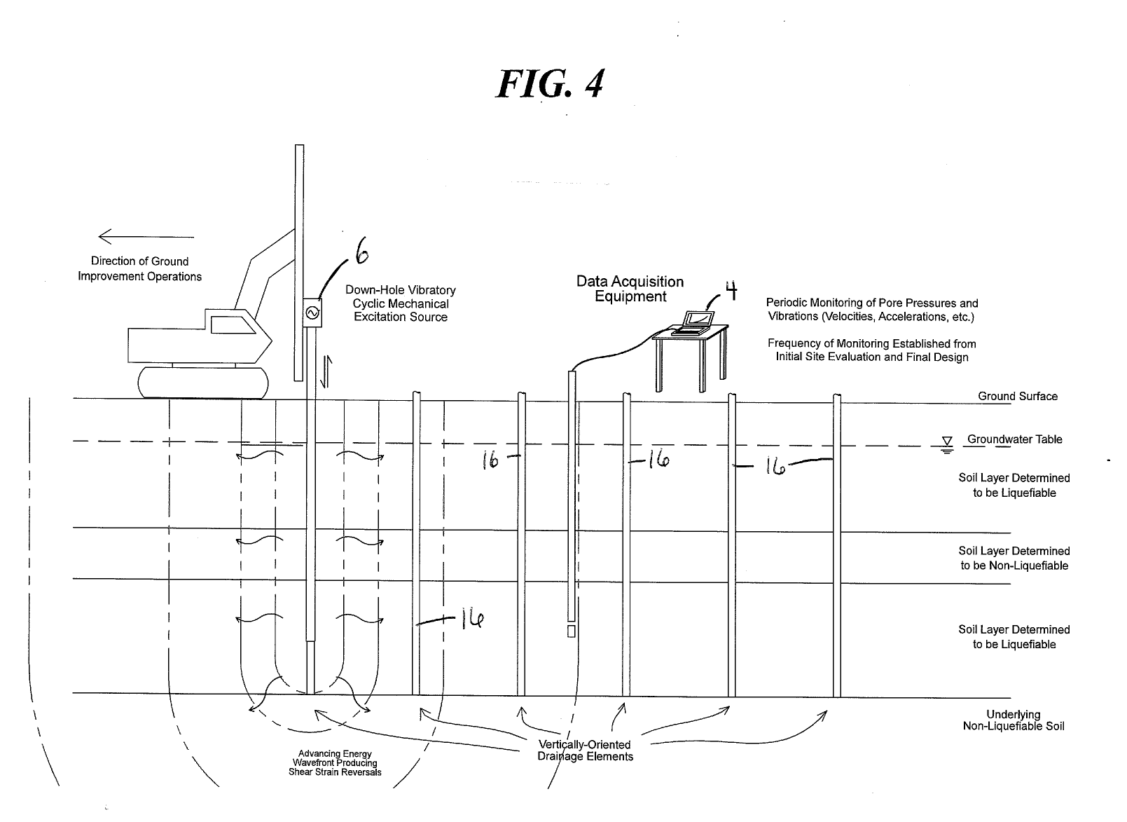

FIG. 4 is an overview of a treatment (ground improvement) of the site using drainage techniques. The pattern and frequency of treatment locations may be determined by frequent monitoring of the in-situ liquefaction sensor 2. During installation of vertically-oriented drainage elements 16, the site may be subjected to shear strain reversals (displacements/velocities/accelerations) in excess of those anticipated from a design earthquake. The pattern, spacing, density and frequency of treatment locations at the site is chosen to ensure that excess pore pressures generated in treated soils during ground improvements do not exceed predetermined acceptable values, whereupon the onset of liquefaction would occur.

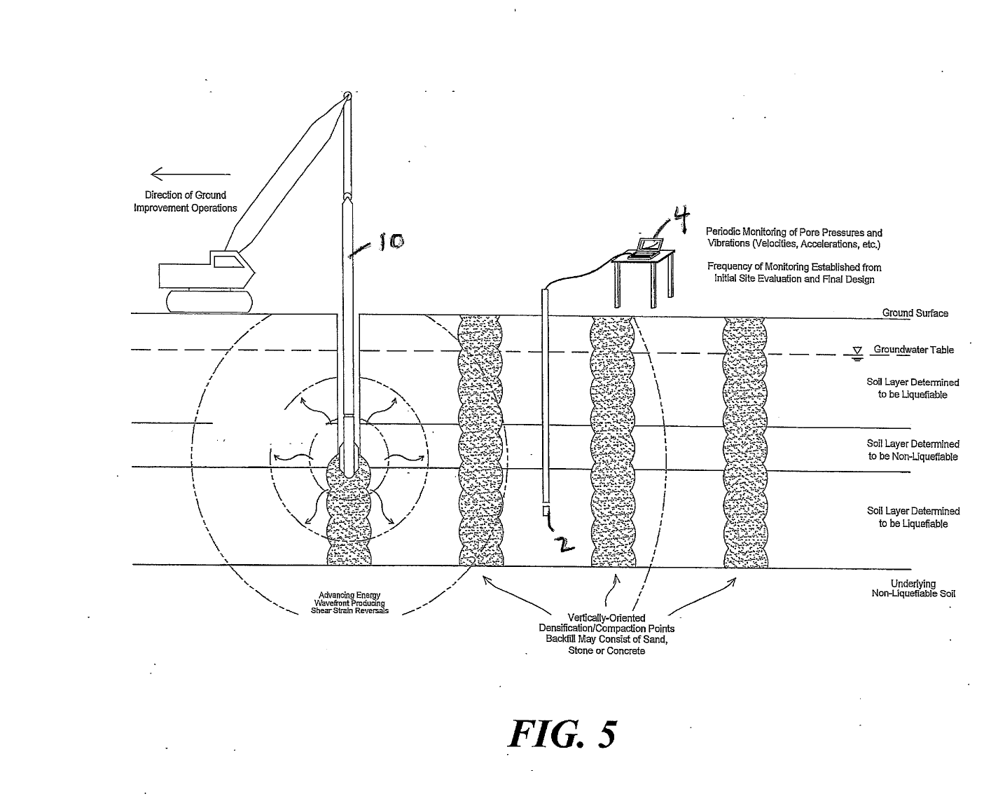

FIG. 5 is an overview of subsequent treatment (ground improvement) of the site using soil densification techniques. The pattern, spacing, density and frequency of treatment locations are determined by frequent monitoring of the in-situ liquefaction sensor. The soils are subjected to shear strain reversals (displacements/velocities/accelerations) in excess of those anticipated from the design earthquake. The pattern spacing, density and frequency of treatment locations is chosen to ensure that excess pore pressures generated in treated soils during the ground improvements do not exceed predetermined acceptable values, whereupon the onset of liquefaction would occur. In this embodiment, densification is achieved in part by employing vibratory equipment 10 that may be applied in generally vertical holes formed in the soil and extending below the water table. Ground improvement by vibratory densification of the site preferably uses sand, stone and/or gravel as backfill materials for the holes so formed.

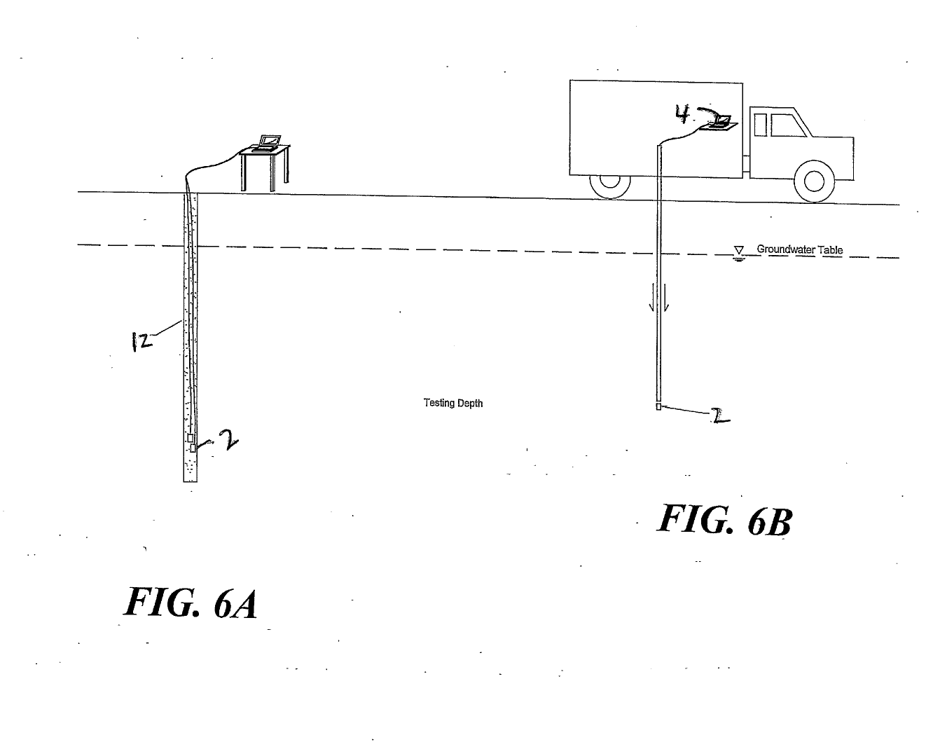

FIGS. 6A and 6B demonstrate methods for placing/installing the liquefaction sensor 2 at a determined appropriate depth for initial site evaluation and subsequent treatment/ground improvements. FIG. 6A illustrates the installation of the liquefaction sensor array at depth in a pre-drilled/pre-bored hole 12. FIG. 6B illustrates the installation of the liquefaction sensor array 2 to the desired testing depth by means of direct push (pneumatic/electric/hydraulic) technology. The data acquisition equipment 4 may be positioned in a vehicle that communicates with the sensor array.

In an embodiment of the invention, the subject site is tested under varying shear strain reversals (soil accelerations), while pore pressure generation is monitored. The actual liquefaction susceptibility of the soil deposit in question (and individual soil layers throughout said deposit) is determined using anticipated design earthquake peak soil accelerations (shear strain reversals).

Subsequent treatment of the site, if necessary, may include installation of additional positive vertical drainage elements, positioned as determined from shear strain using mechanical equipment to generate repeated shear strain reversals (peak soil accelerations) equivalent to or greater than those anticipated by the design earthquake through the full depth of the liquefiable soils (while measuring pore pressure generation and dissipation) for periods of time exceeding those anticipated from the design earthquake.

The mechanical excitation equipment employed according to the methods described herein is preferred to generate soil accelerations (shear strain reversals) in the range of Ag to 2 g and in a 2 to 50 Hz frequency domain. The mechanical excitation equipment is preferred to produce vibrations in the soil within these ranges. The spacing and frequency of the excitation points and accompanying ground improvement is determined and continuously altered/revised across the project site so that, upon completion of the improvements, all final measured excess generated pore pressures are held to levels less than those at which the onset of liquefaction occurs.

What is claimed is:

1. A method of soil liquefaction testing and remediation, comprising the steps of:
determining that a soil deposit at a site is potentially subject to liquefaction;
subjecting the soil deposit at the site to mechanically produced shear strain reversals;
monitoring excess pore pressure generation at multiple depths of the soil deposit and at multiple locations of the soil deposit;
determining actual liquefaction susceptibility of the soil deposit based on pore pressure response to mechanically produced shear strain reversals;
positioning a plurality of spaced apart ground improvements on the site;
subjecting the site to mechanically produced shear strain reversals subsequent to positioning the plurality of spaced apart ground improvements on the site; and
measuring excess pore pressure generation while subjecting the site to mechanically produced shear strain reversals subsequent to positioning the plurality of spaced apart ground improvements on the site.

2. A method of soil liquefaction testing and remediation as described in claim 1, comprising the additional steps of:
installing testing equipment prior to subjecting the soil deposit to mechanically produced shear strain reversals, wherein the testing equipment is constructed and arranged to measure in-situ pore pressures and soil accelerations at the multiple depths of the soil deposit and the multiple soil locations of the soil deposit.

3. A method of soil liquefaction testing and remediation as described in claim 1, comprising the additional steps of:
installing testing equipment prior to subjecting the soil deposit to mechanically produced shear strain reversals, wherein the testing equipment is constructed and arranged to measure in-situ pore pressures and soil accelerations and the testing equipment is positioned within the soil deposit by direct push technology.

4. A method of soil liquefaction testing and remediation as described in claim 2, wherein the testing equipment is constructed and arranged to measure in-situ pore pressures in a range of 0.0 to 100.0 pounds per square inch (psi).

5. A method of soil liquefaction testing and remediation as described in claim 2, wherein the testing equipment is constructed and arranged to measure in-situ soil accelerations in a range of 0.1 to 2.0 g.

6. A method of soil liquefaction testing and remediation as described in claim 1, wherein the mechanically produced shear strain reversals are produced by soil accelerations, wherein the soil accelerations are equal to or greater than accelerations of a design earthquake at the site, and a duration of soil accelerations continues for a period of time that exceeds a theoretical time of the design earthquake chosen for the site.

7. A method of soil liquefaction remediation as described in claim 1, wherein the mechanically produced shear strain reversals are produced by soil accelerations, and the soil accelerations are in a range of 0.1 g to 2.0 g and the frequency domain of the soil accelerations is in the range of 2 to 50 Hz.

8. A method of soil liquefaction remediation as described in claim 1, wherein the step of determining that the soil deposit on the site is potentially subject to liquefaction is performed using data available from an initial geotechnical investigation and from shear strain reversal and pore pressure generation and dissipation data collected while the soil deposit is subjected to mechanically produced shear strain reversals.

9. A method of soil liquefaction remediation as described in claim 1, wherein positioning of the plurality of spaced apart ground improvements is determined by evaluation of shear strain reversals and pore pressure generation and dissipation data obtained at multiple locations at the site taken at multiple soil depths.

10. A method of soil liquefaction remediation as described in claim 1, wherein positioning of the plurality of spaced apart ground improvements is revised and altered subsequent to an initial positioning of the plurality of spaced apart ground improvements based upon data obtained from measuring excess pore pressure generation while subjecting the site to mechanically produced shear strain reversals produced subsequent to initial positioning the plurality of ground improvements on the site, wherein the shear strain reversals and time durations exceed the time of the theoretical earthquake chosen for the design earthquake for the site.

11. A method of soil liquefaction testing and remediation as described in claim 1, comprising the additional steps of:
installing testing equipment prior to subjecting the soil deposit to mechanically produced shear strain reversals, wherein the testing equipment is constructed and arranged to measure in-situ pore pressures and soil accelerations and the testing equipment is positioned at a depth in the soil deposit by placement into a drilled borehole.

12. A method of soil liquefaction testing and remediation described in claim 1, wherein the plurality of spaced apart ground improvements comprises a plurality of spaced apart positive drainage elements inserted to a depth in the soil deposit below a water table at the site.

13. The method of soil liquefaction testing and remediation described in claim 1, wherein the mechanically produced shear strain reversals are produced by vibratory equipment that produces soil acceleration by means of energy waves.

14. The method of soil liquefaction testing and remediation described in claim 1, wherein the mechanically produced shear strain reversals are produced by vibratory equipment operating in a range of 0.1 g to 2.0 g and in a frequency domain in the range of 2 to 50 Hz.

15. The method of soil liquefaction testing and remediation described in claim 1, wherein the site is subjected to shear strain reversals during installation of ground improvements.

16. A method of soil liquefaction testing and remediation as described in claim 1, comprising the additional steps of:
installing testing equipment prior to subjecting the soil deposit to mechanically produced shear strain reversals, wherein the testing equipment is constructed and arranged to measure in-situ pore pressures and soil accelerations in up to three dimensions.
